(19) United States
(12) Patent Application Publication
(10) Pub. No.: US 2024/0050654 A1
(43) Pub. Date: Feb. 15, 2024

(54) INTRAVENOUS INFILTRATION SENSOR DEVICE

(71) Applicant: Murata Manufacturing Co., Ltd., Nagaokakyo-shi, Kyoto-fu (JP)

(72) Inventors: Koji YOSHIDA, Smyrna, GA (US); Chris MCGUILL, Smyrna, GA (US)

(21) Appl. No.: 17/766,352

(22) PCT Filed: Sep. 28, 2020
(86) PCT No.: PCT/US2020/053008

§ 371 Date: Apr. 4, 2022

Related U.S. Application Data
(60) Provisional application No. 62/924,295, filed on Oct. 22, 2019.

(51) Int. Cl. A61M 5/168

(52) U.S. Cl. CPC A61M 5/16836; A61M 2230/65; A61M 2205/0233; A61M 2205/3553; A61M 2205/50; A61M 2205/8206; A61M 2205/0216; A61M 2005/1586

(57) ABSTRACT

A sensor device includes a sensor, and a plurality of sensor electrodes including a first sensor electrode and a second sensor electrode that are electrically connected to the sensor. Each of the plurality of sensor electrodes can include an inner electrode and an outer electrode surrounding the inner electrode. A first outer electrode of the first sensor electrode and a second outer electrode of the second sensor electrode can detect a current applied between the first outer electrode and the second outer electrode. A first inner electrode of the first sensor electrode and a second inner electrode of the second sensor electrode can detect a voltage applied between the first inner electrode and the second inner electrode.

### BACKGROUND OF THE INVENTION

### 1. Field of the Invention

The present invention relates to intravenous (IV) infiltration sensor devices.

### 2. Background

Intravenous (i.e., within a vein) refers to administering medications or fluids into a patient's vein via a needle, syringe, or tube (catheter). This allows the medicine or fluid to enter the patient's bloodstream immediately.

IV infiltration is one of the most common problems that can occur when IV fluid or medications accidentally leak or infuse into the tissues surrounding an IV site. This can be caused by improper placement or dislodgment of the needle or catheter when the tip of the catheter slips out of the vein. Patient movement can cause the catheter to slip out or through the blood vessel, pass through the wall of the vein, or the blood vessel wall allows some of the fluid to infuse into the surrounding tissue.

Similarly, extravasation occurs when there is accidental infiltration of a vesicant or chemotherapeutic drug into the surrounding IV site. This can cause tissue damage or blistering that can lead to pain, delayed healing, infection, disfigurement, and loss of patient mobility.

Periodic observation, inspection, and treatment of the IV site by a health care provider can help minimize or prevent many of the complications associated with IV infiltration, which can include visual assessment, palpation of the IV site and surrounding tissue, and review of the infusion flow rate.

To assist health care providers, electronic sensors have been developed to automatically detect changes around the IV site and set an alarm or notification of a potential problem. Some sensors monitor the changes in the optical properties of the tissue at the IV site where the amount of light detected by the sensor mostly depends on the scattering and absorption properties of the monitored tissue. Some sensors use ultrasound to detect tissue changes. Other sensors monitor physiological changes in the tissue at the IV site, such as localized body temperature or bioimpedance with electrodes. IV fluid in the patient's tissue changes the electrical impedance properties of the patient's tissue. Thus, an impedance change of a certain level of the patient's tissue in the vicinity of the IV site can be interpreted as being caused by infiltration or extravasation.

U.S. Pat. No. 6,408,204 provides four in-line electrodes on a sheet that is attached to the skin that detects an impedance change of the patient's tissue. The outer two electrodes of the four in-line electrodes are used to provide a current, and the inner two electrodes of the four in-line electrodes detect changes in voltage. With this arrangement of the four in-line electrodes, the detection area is limited to be located between the two inner electrodes. This type of structure minimizes the detection area over the patient's skin and limits freedom to improve the electronic design.

### SUMMARY OF THE INVENTION

To overcome the problems and to satisfy the needs described above, preferred embodiments of the present invention are directed to intravenous infiltration sensor devices that each sense over a wider area and provide greater design freedom.

According to a preferred embodiment of the present invention, a sensor device includes a sensor, and a plurality of sensor electrodes including a first sensor electrode and a second sensor electrode that are electrically connected to the sensor. Each of the plurality of sensor electrodes can include an inner electrode and an outer electrode surrounding the inner electrode. A first outer electrode of the first sensor electrode and a second outer electrode of the second sensor electrode can detect a current applied between the first outer electrode and the second outer electrode. A first inner electrode of the first sensor electrode and a second inner electrode of the second sensor electrode can detect a voltage applied between the first inner electrode and the second inner electrode.

The sensor device can further include a substrate including an opening, wherein the sensor is on the substrate, and the first sensor electrode and the second sensor electrode are on the substrate and are located about the opening.

The substrate can be flexible and can include an adhesive. The sensor can be detachable from the substrate. The device can further include a socket attached to the substrate to which the sensor is attached.

The plurality of sensor electrodes can be three or four sensor electrodes. Each of the plurality of sensor electrodes can be located equidistant from the opening. The plurality of sensor electrodes can include a single-structure electrode.

The sensor communicates with a remote monitor. The device can further include an IV stabilization device.

The device can further include wiring that is located on a single side of the substrate and that connects the sensor and the plurality of sensor electrodes. The device can further include wiring that is located on opposing sides of the substrate and that connects the sensor and the plurality of sensor electrodes.

The sensor and the plurality of sensor electrodes can be on a same side of the substrate. The sensor can be located in a first region of the substrate, the plurality of sensor electrodes can be located in a second region of the substrate separate from the first region, and the first and second regions can be divided along a folding line of the substrate.

The opening can define an intravenous hole. The substrate can have a U-shape that defines the opening. The sensor can detect intravenous infiltration or extravasation.

According to a preferred embodiment of the present invention, a substrate for adhering to a patient's skin can include a socket that receives a sensor, first and second sensor electrodes, a first wiring that connects the first sensor electrode to the socket, and a second wiring that connects the second sensor electrode to the socket. The first sensor electrode includes a first inner electrode and a first outer electrode surrounding the first inner electrode. The second sensor electrode includes a second inner electrode and a second outer electrode surrounding the second inner electrode. The first outer electrode of the first sensor electrode and the second outer electrode of the second sensor electrode detect a current applied between the first outer electrode and the second outer electrode. The first inner electrode of the first sensor electrode and the second inner electrode of the second sensor electrode detect a voltage applied between the first inner electrode and the second inner electrode.

The substrate can further include an opening. The opening can define an intravenous hole.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

FIG. 1 shows an intravenous infiltration sensor device and a remote monitoring device according to a first preferred embodiment of the present invention.

FIGS. 2 and 3 show bottom views of an intravenous infiltration sensor device.

FIGS. 4 and 5 are schematic circuit diagrams of electrodes of an intravenous infiltration sensor device.

FIGS. 6A and 6B are bottom views of an intravenous infiltration sensor device according to a second preferred embodiment of the present invention.

FIG. 7 is a schematic circuit diagram of electrodes of an intravenous infiltration sensor device.

FIGS. 8 and 9 show an intravenous infiltration sensor device according to a third preferred embodiment of the present invention.

FIGS. 10 and 11 show another aspect of the intravenous infiltration sensor device according to the third preferred embodiment of the present invention.

FIGS. 12 and 13 show an intravenous infiltration sensor device according to a fourth preferred embodiment of the present invention.

FIGS. 14 and 15 show another aspect of the intravenous infiltration sensor device according to the fourth preferred embodiment of the present invention.

FIG. 16 is a schematic circuit diagram of electrodes of an intravenous infiltration sensor device according to the fourth preferred embodiment of the present invention.

FIG. 17 shows an intravenous infiltration sensor device according to a fifth preferred embodiment of the present invention.

FIGS. 18 and 19 show another aspect of the intravenous infiltration sensor device according to the fifth preferred embodiment of the present invention.

### DETAILED DESCRIPTION OF PREFERRED EMBODIMENTS

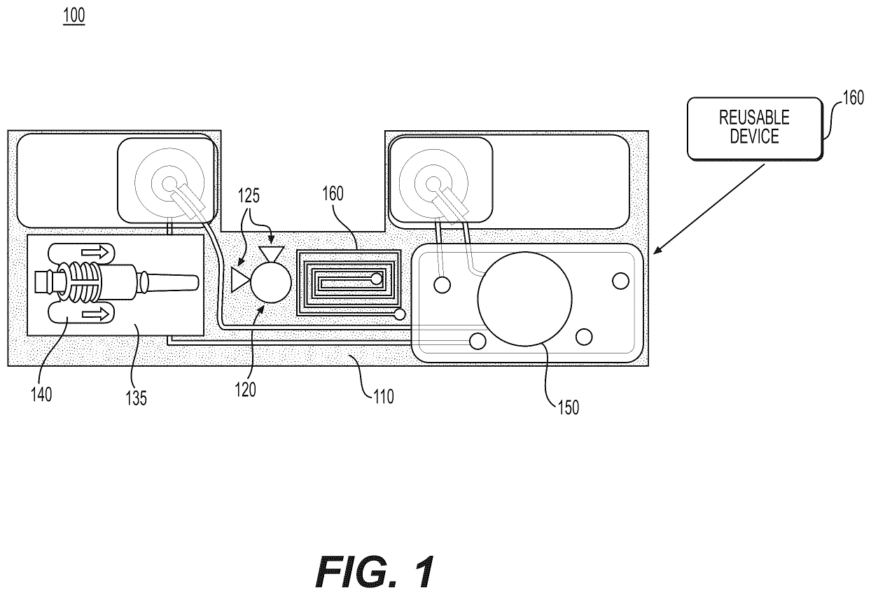

FIG. 1 shows an intravenous infiltration sensor device 100. The device 100 can include a flexible substrate 110 with an opening 120 used as an IV hole that defines a pathway to insert a needle or catheter into a patient's skin, stabilization marks 125 as guides for placing the needle or catheter through the opening 120, an IV stabilization device 130 supported by a foam stiffener 135 to help secure and stabilize the needle or catheter and tubing during use, and electronic sensor components. FIG. 1 shows that the electronic sensor components can include a sensor 140 that is attached to the substrate 110 and that can communicate with a remote monitoring device (not shown), a replaceable battery 150 attached to the substrate 110 as a source to power the sensor 140, and an antenna 160 that can be included in the substrate 110 and that can be used to transmit or receive signals between the sensor 140 and the remote monitoring device. Although shown as separate components in FIG. 1, the sensor 140, replaceable battery 150, and antenna 160 can be included in a single removable sensor. The substrate 110 can include a socket that receives the sensor 140. The replaceable battery 150 can be included in the socket. The sensor 140 can include one or more of a microcontroller unit (MCU), Bluetooth low energy (BLE) module, a light-emitting diode (LED), circuitry to analyze bioimpedance, and a front-end circuit connected to the antenna. The circuitry to analyze the bioimpedance can be an integrated circuit (IC) or other similar device. The front-end circuit can process the signals received by the antenna and can be a custom analog front-end circuit or other similar device. Alternatively, the device 100 can include a reusable sensor device 160, as further discussed below.

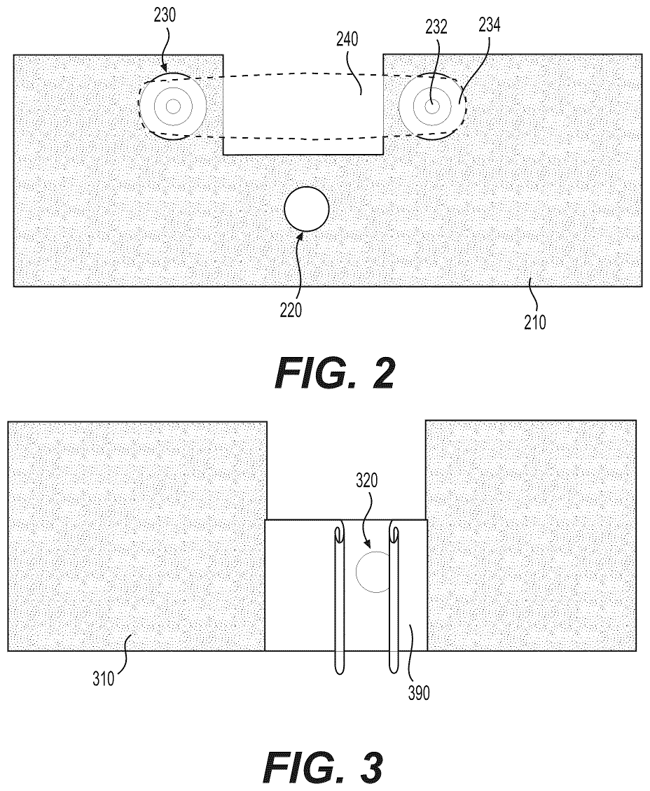

FIG. 2 is a bottom view of an intravenous infiltration sensor device. FIG. 2 shows a substrate 210 that can be a flexible and stretchable material to be attached to a patient's skin. An IV hole 220 can be included in the substrate 210. FIG. 2 also shows two sensor electrodes 230 on opposite sides of a detection area 240, represented as the area within the dotted line. The sensor electrodes 230 can include an inner electrode 232 and an outer electrode 234 that can completely surround the inner electrode 232. Although, FIG. 2 shows the inner electrode 232 and the outer electrode 234 as circular, other geometric shapes are possible. In operation, a sensor measures physiological signals of the patient's skin in the detection area 240 between the sensor electrodes 230. For example, the sensor can measure the voltage and the impedance of the detection area.

FIG. 3 is a bottom view of an intravenous infiltration sensor device including an IV hole 320. FIG. 3 also shows that the substrate 310 can include an adhesive (not shown) used to adhere the substrate 310 to the patient's skin. Also, the substrate 310 can include a separator 390 or release film that covers the adhesive during storage. The separator 390 can be peeled away to expose the adhesive on the substrate 310 prior to application of the substrate 310 to the patient's skin similar to the method associated with applying a common adhesive bandage. The separator 390 can be divided at the IV hole 320 as shown in FIG. 3, but the separator 390 can be divided at any other location as well.

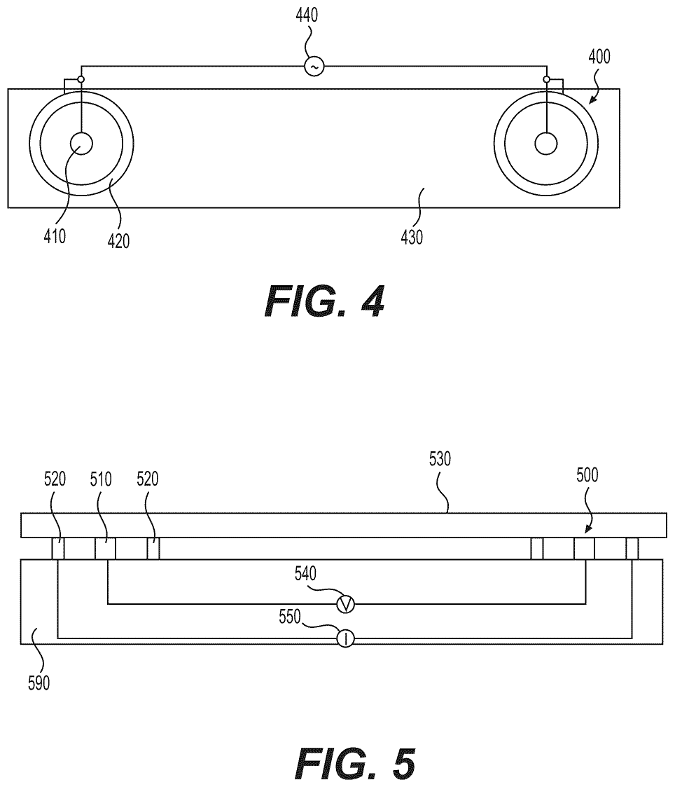

FIGS. 4 and 5 are schematic circuit diagrams of electrodes of an intravenous infiltration sensor device. FIG. 4 shows a configuration of two sensor electrodes 400 separated by a distance, where both sensor electrodes 400 have a double-electrode structure including an inner electrode 410 and an outer electrode 420 that are on a bottom of the substrate 430 and adhered to a patient's skin. As shown, the double-electrode structure of the sensor electrode 400 can be defined with the inner electrode 410 as a dot of conductive material surrounded by a ring of conductive material as the outer electrode 420. A sensor 440 is used to sense bioimpedance of the patient's skin between the two sensor electrodes 400, as shown in FIG. 5. The sensor 440 can provide a signal with a predetermined voltage and a predetermined current.

FIG. 5 shows a cross section of the electrodes of an intravenous infiltration sensor device on a patient's skin 590. FIG. 5 shows two double-structure sensor electrodes 500 separated by a distance and including the inner electrode 510 and the outer electrode 520 on the substrate 530. The intravenous infiltration sensor device measures bioimpedance parameters of the patient's skin 590 between the sensor electrodes 500, including, for example, voltage 540 between the inner electrodes 510 and current 550 between the outer electrodes 520. It is also possible that the outer electrodes 520 measure voltage 540 and the inner electrodes 510 measure current 550. Changes in the voltage 540 and the current 550 between the two sensor electrodes 500 can be used to detect IV infiltration or extravasation.

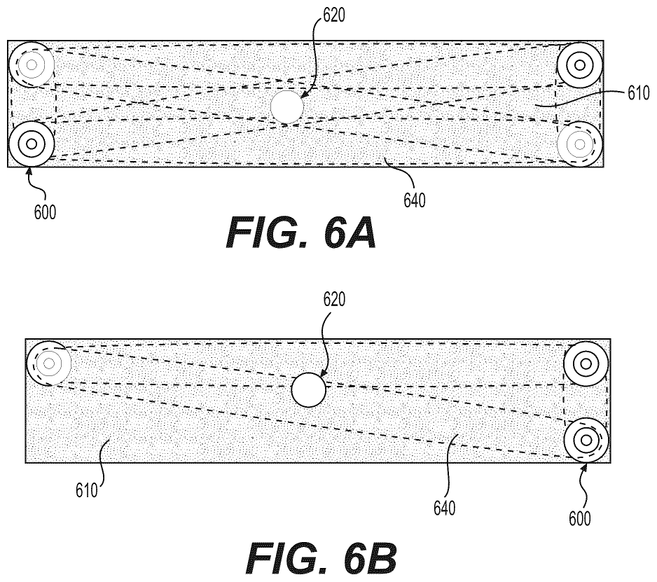

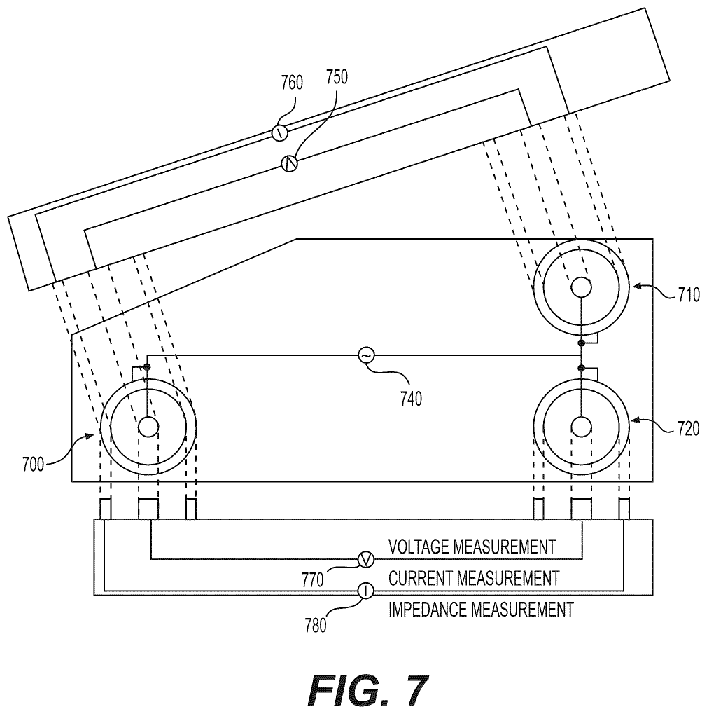

FIGS. 6A and 6B are bottom views of an intravenous infiltration sensor device according to a second preferred embodiment of the present invention with two alternate double-structure sensor electrode 600 configurations. Conductive traces are not shown on the substrates for clarity. FIG. 6A shows a substrate 610, an IV hole 620, and four double-structure sensor electrodes 600 at corners of a detection area 640, shown as an area within the dotted lines. FIG. 6B shows the substrate 610, the IV hole 620, and three double-structure sensor electrodes 600 defining the detection area 640, shown as an area within the dotted lines. As shown, each of the double-structure sensor electrodes 600 can be equidistant from the IV hole 620. Although FIGS. 6A and 6B show configurations with three and four double-structure sensor electrodes 600 respectively, other numbers of sensor electrodes, electrode structures, and physical layouts are possible. Accordingly, more than two sensor electrodes can be used to sense bioimpedance of a patient's skin adjacent to an IV site over a greater area than with a configuration using two sensor electrodes, as described in more detail with respect to FIG. 7.

FIG. 7 is a schematic circuit diagram of electrodes of the intravenous infiltration sensor device. FIG. 7 shows a configuration of three double-electrode sensor electrodes 700, 710, and 720 separated by distances, similar to that shown in FIG. 6B and operated similarly as that described with respect to FIGS. 4 and 5. In this configuration, a sensor 740 is used to sense bioimpedance of the patient's skin between the three sensor electrodes 700, 710, and 720. As shown in FIG. 7, a voltage 750 and a current 760 can be measured between the sensor electrodes 700 and 710, and also a voltage 770 and a current 780 can be measured between the sensor electrodes 700 and 720. Although not shown in FIG. 7, a voltage and a current can be measured between the sensor electrodes 710 and 720. Changes in the voltage 750 or 770 and the current 760 or 780 between the two sensor electrodes 700 and 710 or 720 can be used to detect IV infiltration or extravasation.

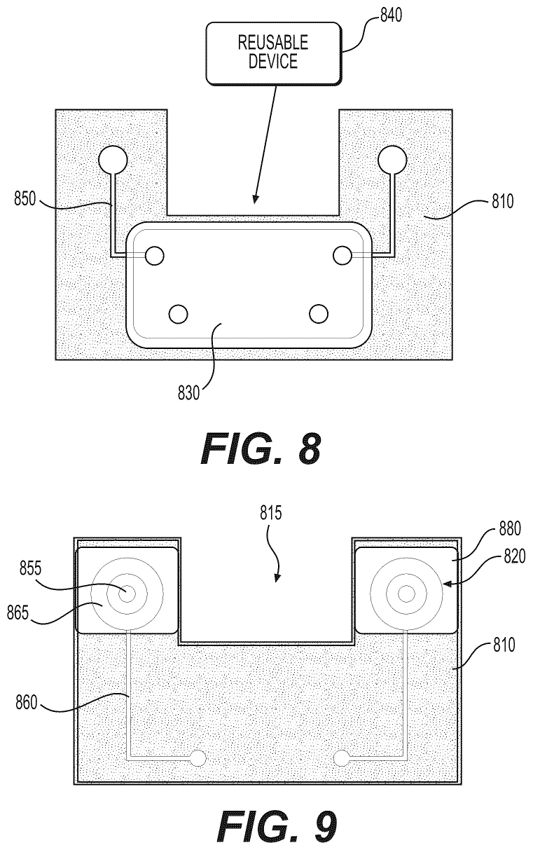

FIGS. 8 and 9 show an intravenous infiltration sensor device according to a third preferred embodiment of the present invention. FIG. 8 shows a top view and FIG. 9 shows a bottom view of the intravenous infiltration sensor device. As shown in FIGS. 8 and 9, the intravenous infiltration sensor device can include a U-shaped flexible substrate 810 with an adhesive on the bottom (not shown) to attach the intravenous infiltration sensor device to a patient's skin. The opening 815 of the U-shape is meant to be placed around an IV site such that the double-electrode sensor electrodes 820 are on two sides of the IV site. Although a U-shaped flexible substrate 810 is shown in FIGS. 8 and 9, it is possible to use other shapes. For example, the substrate 810 could be rectangular with an opening in the middle such that substrate 810 surrounds the IV site on all sides. A conductive material such as a gel or spray 880 can be used to facilitate electrical contact between the sensor electrodes 820 and the patient's skin. It is also possible to use a dry electrode. Although not shown in FIGS. 8 and 9, it is possible to include an IV stabilization device on the substrate 810, for example, by including the IV stabilization device instead of the opening 815. Although only two double-electrode sensor electrodes 820 are shown in FIG. 9, three, four, or more double-electrode sensor electrodes can be used.

FIG. 8 shows a socket 830 is provided on the substrate 810 used to electrically and physically connect a reusable sensor device 840 to the substrate 810. The socket 830 can be a header, connector, or any other device suitable for keeping the reusable sensor device 840 and the substrate 810 in contact during use and for allowing disconnection of the reusable sensor device 840 from the substrate 810 while not in use. The reusable sensor device 840 can include a power supply, i.e., a battery, and electronic sensor components used to measure bioimpedance and communicate with a processing device or monitor. The reusable sensor device 840 can be removed and the substrate 810 can be disposed of after use.

FIG. 8 also shows top-side wiring 850 that provides electrical connection by conductive traces and vias between an inner electrode 855 of the sensor electrode 820 and the socket 830. FIG. 9 shows bottom-side wiring 860 that provides electrical connection by conductive traces and vias between an outer electrode 865 of the sensor electrode 820 and the socket 830. In the wiring configurations shown in FIGS. 8 and 9, the conductive traces can be formed by screen printing, jet printing of conductive ink, or any other suitable method. A laser can be used to cut holes in the substrate that are then filled with a metal or other conductive material to define vias through the substrate to connect the wiring and the sensor electrodes that are on opposite sides of the substrate. Using a double-electrode structure allows the intravenous infiltration sensor device to be smaller, while maintaining the same size detection area. Additionally, the substrate can be a thick insulating material that isolates the conductive traces and the electrodes on opposite sides of the substrates and that reduces, minimizes or eliminates electrical short circuits between the conductive traces and the electrodes on opposite sides of the substrates.

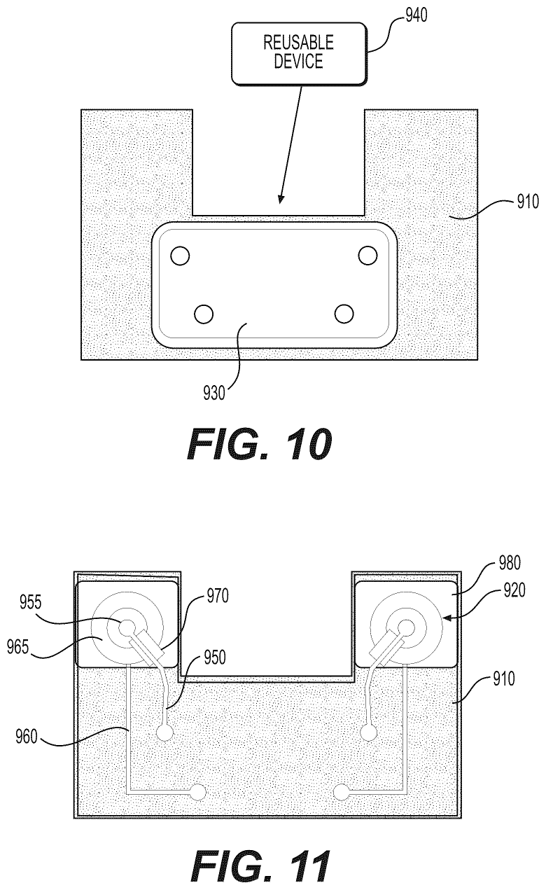

FIGS. 10 and 11 show another aspect of the intravenous infiltration sensor device according to the third preferred embodiment of the present invention. FIG. 10 shows a top view and FIG. 11 shows a bottom view of the intravenous infiltration sensor device. Similarities between the configuration shown in FIGS. 8 and 9 will be omitted for brevity. FIGS. 10 and 11 show a flexible substrate 910, two sensor electrodes 920, a socket 930, and a reusable sensor device 940. FIG. 11 shows all of the wiring 950 and 960 is routed on the bottom side of the intravenous infiltration sensor device between the inner electrode 955 and the outer electrode 965 of the sensor electrode 920 to the socket 930 by conductive traces and vias through the substrate 910. Routing of wiring 950 between the inner electrode 955 and the sensor 930 is made possible on the bottom side of the intravenous infiltration sensor device because an insulator 970 is included between the wiring 950 and the outer electrode 965 that allows the wiring 950 to cross over the outer electrode 965 without short circuiting. This configuration simplifies the process to fabricate the substrate 910 with the sensor electrodes 920 and corresponding wiring 950 and 960 on one side of the substrate 910 that eliminates having traces on both sides of the substrate 910. A conductive material such as a gel or spray 980 can be used to facilitate electrical contact between the sensor electrodes 920 and the patient's skin. It is also possible to use a dry electrode.

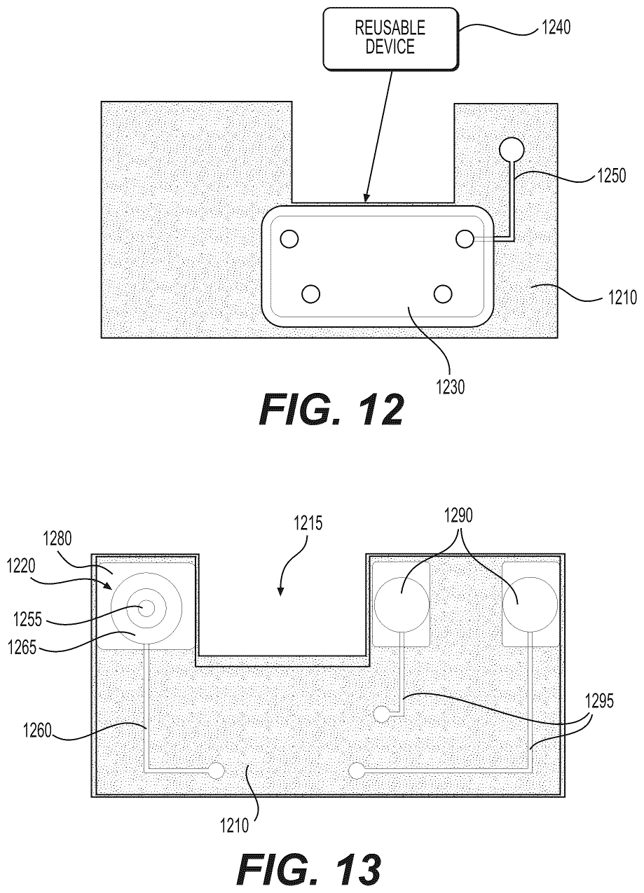

FIGS. 12 and 13 show an intravenous infiltration sensor device according to a fourth preferred embodiment of the present invention. FIG. 12 shows a top view and FIG. 13 shows a bottom view of the intravenous infiltration sensor device with a combination of a double-structure electrode 1220 and two single-structure electrodes 1290. Similarities between the configurations shown in FIGS. 8-11 will be omitted for brevity. FIGS. 12 and 13 show a flexible substrate 1210, a socket 1230, and a reusable sensor device 1240. As shown in FIGS. 12 and 13, the open portion 1215 of the U-shape is offset from the center of the substrate 1210. This offset allows for a greater area of the substrate 1210 on one side of the opening 1215 that includes two single-structure sensor electrodes 1290. The single-structure electrodes 1290 each include one conductive area. As shown in FIG. 13, one double-structure electrode 1220 is on the opposite side of the opening 1215 from the two single-structure electrodes 1290. A conductive material such as a gel or spray 1280 can be used to facilitate electrical contact between the sensor electrodes 1220, 1290 and the patient's skin. It is also possible to use a dry electrode.

FIG. 12 also shows top-side wiring 1250 that provides electrical connection by conductive traces and vias between an inner electrode 1255 of the sensor electrode 1220 and the socket 1230. FIG. 13 shows bottom-side wiring 1260 that provides electrical connection by conductive traces and vias between an outer electrode 1265 of the sensor electrode 1220 and the socket 1230. FIG. 13 also shows wiring 1295 that provides electrical connection by traces and vias between the electrodes 1290 and the socket 1230. As shown in FIG. 13, the wiring 1295 can be located on the bottom of the intravenous infiltration sensor device, but it is also possible that the wiring 1295 is on the top of the intravenous infiltration sensor device.

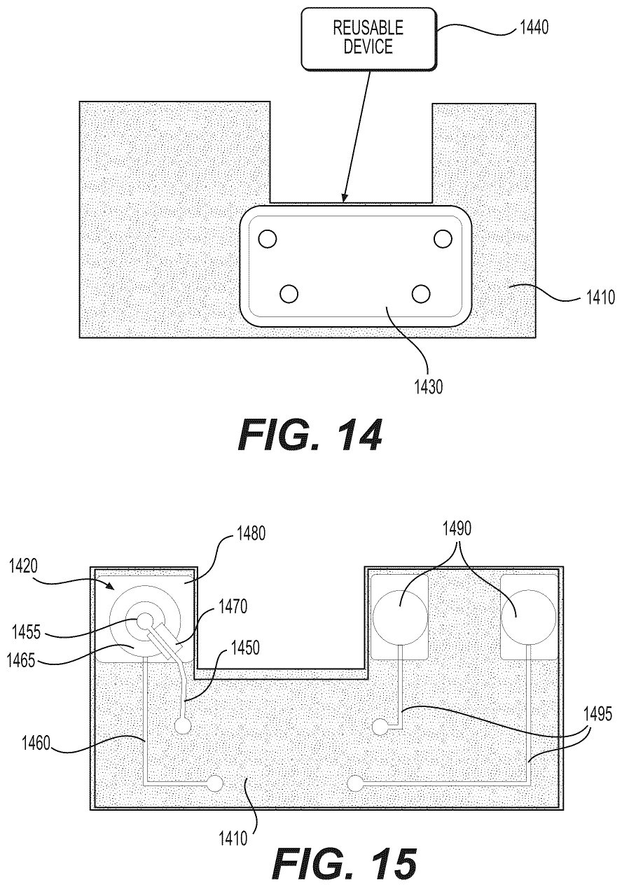

FIGS. 14 and 15 show another aspect of the intravenous infiltration sensor device according to the fourth preferred embodiment of the present invention. FIG. 14 shows a top view and FIG. 15 shows a bottom view of the intravenous infiltration sensor device. Similarities between the configurations shown in FIGS. 8-13 will be omitted for brevity. FIGS. 14 and 15 show a substrate 1410, two single-structure sensor electrodes 1490 with corresponding wiring 1495, a socket 1430, and a reusable sensor device 1440. FIG. 15 shows all of the wiring 1450 and 1460 is routed on the bottom side of the intravenous infiltration sensor device between the inner electrode 1455 and the outer electrode 1465 of the sensor electrode 1420 to the socket 1430 by conductive traces and vias through the substrate 1410. Routing of wiring 1450 between the inner electrode 1455 and the sensor 1430 is made possible on the bottom side of the intravenous infiltration sensor device because an insulator 1470 is included between the wiring 1450 and the outer electrode 1465 that allows the wiring 1450 to cross over the outer electrode 1465 without short circuiting. This configuration simplifies the process to fabricate the substrate 1410 with the sensor electrodes 1420 and corresponding wiring 1450 and 1460 on one side of the substrate 1410 that eliminates the need for conductive vias. A conductive material such as a gel or spray 1480 can be used to facilitate electrical contact between the sensor electrodes 1420, 1490 and the patient's skin. It is also possible to use a dry electrode.

FIG. 16 is a schematic circuit diagram of electrodes of an intravenous infiltration sensor device. FIG. 16 shows the configuration with one double-electrode sensor electrode 1620 separated by a distance between the two single-electrode sensor electrodes 1690 and 1692 similar to that shown in FIGS. 13 and 15 and operated similarly as that described with respect to FIGS. 4, 5, and 7. In this configuration, a sensor 1640 is used to sense bioimpedance of the patient's skin between the three sensor electrodes 1620, 1690, and 1692. As shown in FIG. 16, a voltage 1650 is measured between the inner electrode 1655 of sensor electrode 1620 and sensor electrode 1690, and a current 1660 is measured between the outer electrode 1665 of sensor electrode 1620 and sensor electrode 1692. Changes in the voltage 1650 between the two sensor electrodes 1620, 1690 and changes in the current 1660 between the two sensor electrodes 1620, 1692 can be used to detect IV infiltration or extravasation.

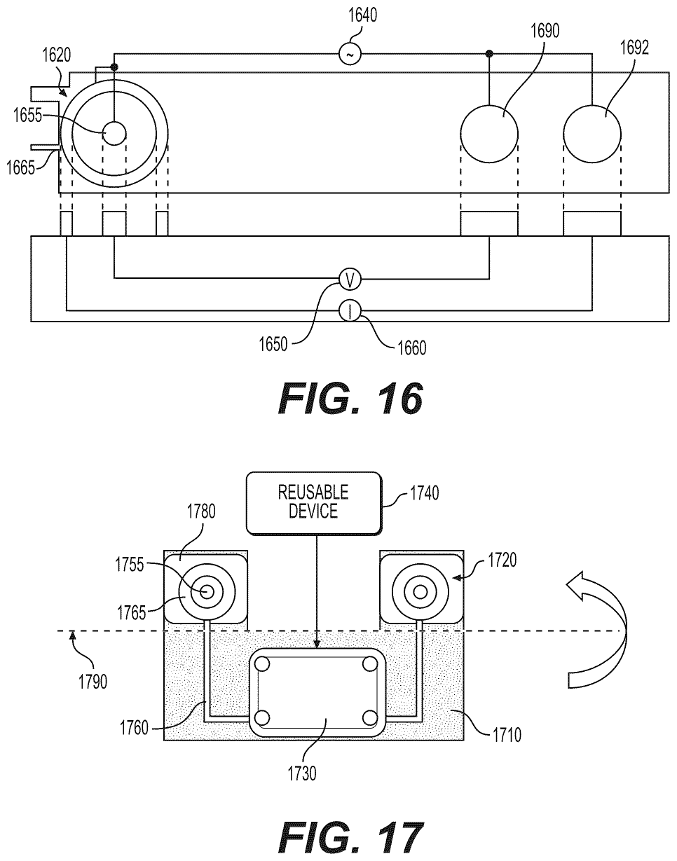

FIG. 17 shows an intravenous infiltration sensor device according to a fifth preferred embodiment of the present invention. Similarities between the configurations shown in FIGS. 8-15 will be omitted for brevity. FIG. 17 shows a substrate 1710, two double-structure sensor electrodes 1720 with one each on either side of the U-shaped opening in the substrate 1710, a socket 1730, and a reusable sensor device 1740. A conductive material such as a gel or spray 1780 can be used to facilitate electrical contact between the sensor electrodes 1720 and the patient's skin. It is also possible to use a dry electrode. Although two sensor electrodes 1720 are shown in FIG. 17, other sensor electrode configurations are possible, including, for example, using three, four, or more sensor electrodes or using one double-electrode sensor electrode and two single-electrode sensor electrodes.

In the arrangement shown in FIG. 17, the sensor electrodes 1720, the wiring 1760 connected to the outer electrode 1765, and the socket 1730 are all on the same side of the substrate 1710. Wiring for the inner electrode 1755 is on the opposite side of the substrate 1710 (not shown). The substrate 1710 can be folded substantially along a folding line 1790, represented by the dotted line and in a direction indicated by the arrow on the right side of FIG. 17, to contact the sensor electrodes 1720 to the patient's skin, while the sensor 1730 and the reusable sensor device 1740 are exposed.

Folding the substrate 1710 reduces the total area of intravenous infiltration sensor device. Thus, it is possible to maintain the size of sensor electrodes 1720, even though the operational size of the intravenous infiltration sensor device is reduced. As a result, the socket 1730 and the reusable sensor device 1740 do not hinder physical and electrical contact of the sensor electrodes 1720 contact with the patient's skin that is flexible. That is, the stiffness of the socket 1730 and the reusable sensor device 1740 does not interfere with establishing acceptable physical and electrical contact between the sensor electrodes 1720 and the patient's skin.

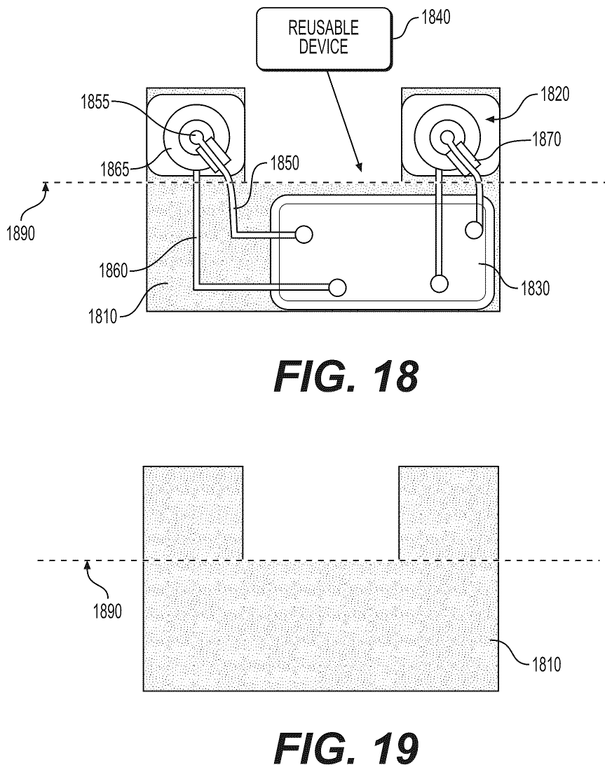

FIGS. 18 and 19 show another aspect of the intravenous infiltration sensor device according to the fifth preferred embodiment of the present invention. FIG. 18 shows a top view and FIG. 19 shows a bottom view of the intravenous infiltration sensor device. Similarities between the configurations shown in FIGS. 8-15 and 17 will be omitted for brevity. FIGS. 18 and 19 show a flexible substrate 1810, two sensor electrodes 1820, a socket 1830, and a reusable sensor device 1840. FIG. 18 shows that all of the inner electrodes 1855, the outer electrodes 1865, and the corresponding wiring 1850 and 1860 are on the same top side of the intravenous infiltration sensor device as the socket 1830 and the reusable sensor device 1840. Routing of wiring 1850 between the inner electrode 1855 and the sensor 1830 is made possible on the same side of the intravenous infiltration sensor device because an insulator 1870 is included between the wiring 1850 and the outer electrode 1865 that allows the wiring 1850 to cross over the outer electrode 1865 without short circuiting.

In this aspect, the substrate 1810 is meant to be folded along the folding line 1890 substantially similar to that shown and described with respect to FIG. 17. In this arrangement, all of the sensor electrodes 1820, the wiring 1850 and 1860, the sensor 1830, and the reusable sensor device 1840 are on the same side of the substrate 1810. As a result, processing of the intravenous infiltration sensor device is simplified.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

1. A sensor device comprising:
a sensor and
a plurality of sensor electrodes including a first sensor electrode and a second sensor electrode that are electrically connected to the sensor, wherein
each of the plurality of sensor electrodes includes an inner electrode and an outer electrode surrounding the inner electrode,
a first outer electrode of the first sensor electrode and a second outer electrode of the second sensor electrode detect a current applied between the first outer electrode and the second outer electrode, and
a first inner electrode of the first sensor electrode and a second inner electrode of the second sensor electrode detect a voltage applied between the first inner electrode and the second inner electrode.

2. The device of claim 1, further comprising a substrate including an opening, wherein
the sensor is on the substrate, and
the first sensor electrode and the second sensor electrode are on the substrate and are located about the opening.

3. The device of claim 2, wherein the substrate is flexible and includes an adhesive.

4. The device of claim 2, wherein the sensor is detachable from the substrate.

5. The device of claim 2, further comprising a socket attached to the substrate to which the sensor is attached.

6. The device of claim 1, wherein the plurality of sensor electrodes is four sensor electrodes.

7. The device of claim 1, wherein the plurality of sensor electrodes is three sensor electrodes.

8. The device of claim 2, wherein each of the plurality of sensor electrodes is located equidistant from the opening.

9. The device of claim 1, wherein the plurality of sensor electrodes includes a single-structure electrode.

10. The device of claim 1, wherein the sensor communicates with a remote monitor.

11. The device of claim 1, further comprising an IV stabilization device.

12. The device of claim 2, further comprising wiring that is located on a single side of the substrate and that connects the sensor and the plurality of sensor electrodes.

13. The device of claim 2, further comprising wiring that is located on opposing sides of the substrate and that connects the sensor and the plurality of sensor electrodes.

14. The device of claim 2, wherein the sensor and the plurality of sensor electrodes are on a same side of the substrate.

15. The device of claim 14, wherein:
the sensor is located in a first region of the substrate;
the plurality of sensor electrodes is located in a second region of the substrate separate from the first region; and
the first and second regions are divided along a folding line of the substrate.

16. The device of claim 2, wherein the opening defines an intravenous hole.

17. The device of claim 2, wherein the substrate has a U-shape that defines the opening.

18. The device of claim 1, wherein the sensor detects intravenous infiltration or extravasation.

19. A substrate for adhering to a patient's skin, the substrate including:
a socket that receives a sensor;
first and second sensor electrodes;
a first wiring that connects the first sensor electrode to the socket; and
a second wiring that connects the second sensor electrode to the socket; wherein
the first sensor electrode includes a first inner electrode and a first outer electrode surrounding the first inner electrode,
the second sensor electrode includes a second inner electrode and a second outer electrode surrounding the second inner electrode,
the first outer electrode of the first sensor electrode and the second outer electrode of the second sensor electrode detect a current applied between the first outer electrode and the second outer electrode, and
the first inner electrode of the first sensor electrode and the second inner electrode of the second sensor electrode detect a voltage applied between the first inner electrode and the second inner electrode.

20. The substrate of claim 19, further comprising an opening.

21. The substrate of claim 20, wherein the opening defines an intravenous hole.
